(19) United States
(12) Patent Application Publication
(10) Pub. No.: US 2019/0038312 A1
(43) Pub. Date: Feb. 7, 2019

(54) Specialized Cannula For Trans-Xiphoid Pericardial Procedures

(71) Applicant: Corfigo, Inc., Montclair, NJ (US)

(72) Inventor: Jason Sperling, Upper Saddle, NJ (US)

(21) Appl. No.: 15/759,615

(22) PCT Filed: Sep. 21, 2016
(86) PCT No.: PCT/US16/52845

§ 371 Date: Mar. 13, 2018

Related U.S. Application Data
(60) Provisional application No. 62/222,454, filed on Sep. 23, 2015.

(51) Int. Cl. A61B 17/34; A61B 17/02; A61B 1/267; A61B 1/05

(52) U.S. Cl. CPC A61B 17/3431; A61B 17/0218; A61B 2017/0225; A61B 1/05; A61B 2017/3445; A61B 1/2676

(57) ABSTRACT

A device for facilitating trans-pericardial or sub-xiphoid cardiac procedures includes a flexible primary hollow tube with radii of generally 5-15 mm. The primary hollow tube includes a generally J-shaped curve at a distal end of the primary hollow tube. The primary hollow tube is configured to accommodate an obturator that straightens the primary hollow tube and eliminates the J-shaped curve at the distal end. The J-shaped curve of the primary hollow tube can be manipulated, exaggerated or lessened by an integral mechanism within a wall of the primary hollow tube. A method of placing a primary hollow tube with a generally J-shaped curve at a distal end of the primary hollow tube via sub-xiphoid or trans-pericardial access in order to carry out procedures on a surface of the heart is further disclosed.

### CROSS REFERENCE TO RELATED APPLICATION

This application claims priority to International PCT Application No. PCT/US16/52845, filed on Sep. 21, 2016, which claims priority to U.S. Provisional Application Ser. No. 62/222,454 entitled “Specialized Cannula For Trans-Xiphoid Pericardial Procedures,” filed on Sep. 23, 2015, which is incorporated herein by reference in its entirety.

### BACKGROUND OF THE DISCLOSURE

Typical open-heart procedures are highly invasive and require either division of the breastbone or rib splitting incisions, which are morbid and entail long recovery times. Minimally invasive access to the epicardial (outer) surface of the heart is possible for certain therapeutic cardiac procedures including but not limited to ablation of cardiac tissue to treat rhythm disturbances and ligation of the left atrial appendage to prevent stroke. Some examples are videoscopic access through the ribs via ports or cannulas, and traversing the pericardium (heart sac) and using instruments at a distance, and more recently, laparoscopic access through the abdomen and traversing the diaphragm to gain access to the space immediately around the heart. Access can also be gained just below the lowest extension of the sternum (breastbone), also called the xiphoid process, which is typically cartilaginous. The space underneath the xiphoid can be accessed to open the anterior portion of the pericardium (heart sac), but this grants access to only a very limited portion of the heart, usually the anterior surface of the right ventricle and the edge of the heart called the acute margin. Still, sub-xiphoid access is a less painful approach to the heart because it does not traverse the ribs, breastbone or abdominal muscles.

### SUMMARY OF THE DISCLOSURE

One aspect of the present disclosure is directed to a device for facilitating trans-pericardial or sub-xiphoid cardiac procedures, the device comprising a flexible primary hollow tube with radii of generally 5-15 mm. The primary hollow tube includes a generally J-shaped curve at a distal end of the primary hollow tube. The primary hollow tube is configured to accommodate an obturator that straightens the primary hollow tube and eliminates the J-shaped curve at the distal end.

In some embodiments, the J-shaped curve of the primary hollow tube can be manipulated, exaggerated or lessened by an integral mechanism within a wall of the primary hollow tube. The primary hollow tube may include a secondary inner hollow tube having a diameter 4-8 mm, the secondary inner hollow tube extending along a length of the primary hollow tube. The secondary inner hollow tube may be configured to flex and includes a generally J-shaped curve at a distal end of the secondary inner hollow tube. The secondary inner hollow tube may be configured to allow passage of a flexible videoscope. The flexible videoscope may be coupled to a controller. The obturator may contain a groove to allow the displacement of the secondary inner hollow tube and fits with it in a lock-and-key configuration. The primary hollow tube may be generally flat or oval-shaped. The primary hollow tube may have a cross sectional area between 80 and 300 mm² or a diameter 10-20 mm, and long axis length of 20-30 cm.

Another aspect of the present disclosure is directed to a method of teaching placement of a primary hollow tube with a generally J-shaped curve at a distal end of the primary hollow tube via sub-xiphoid or trans-pericardial access in order to carry out procedures on a surface of the heart.

In some embodiments, the method further may comprise straightening out the primary hollow tube with an obturator to eliminate the J-shaped curve at the distal end. The method further may comprise manipulating, exaggerating or lessening the J-shaped curve of the primary hollow tube by an integral mechanism within a wall of the primary hollow tube. The primary hollow tube may include a secondary inner hollow tube having a diameter 4-8 mm, the secondary inner hollow tube extending along a length of the primary hollow tube. The secondary inner hollow tube may be configured to flex and includes a generally J-shaped curve at a distal end of the secondary inner hollow tube. The secondary inner hollow tube may be configured to allow passage of a flexible videoscope. The flexible videoscope may be coupled to a controller. The method further may comprise securing the obturator in place on the primary hollow tube. The obturator may contain a groove to allow the displacement of the secondary inner hollow tube and fits with it in a lock-and-key configuration. The primary hollow tube may be generally flat or oval-shaped. The primary hollow tube may have a cross sectional area between 80 and 300 mm² or a diameter 10-20 mm, and long axis length of 20-30 cm.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by references signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the disclosure. In the figures:

FIGS. 1A-C are perspective views of a general assembly of a cannula device of one embodiment of the present disclosure;

FIG. 2 is a perspective view of the cannula device being used by placement in a sub-xiphoid location between a bottom of a breastbone and abdomen;

FIG. 3 is a perspective view of a cannula device of another embodiment of the present disclosure; and

FIGS. 4A and 4B are perspective views of a cannula device of yet another embodiment of the present disclosure.

### DETAILED DESCRIPTION OF THE DRAWINGS

In order to use sub-xiphoid access to treat remote areas of the heart, a novel cannula device is described that allows access specifically to the undersurface of the left atrium heart chamber, and also to more remote recesses such as the left atrial appendage. This specialized access complements new procedures and devices meant to access the posterior wall of the left atrium to treat persistent forms of atrial fibrillation, and also opens up opportunities to treat other areas of the heart (left atrial appendage and surface locations of the left ventricle with foci of dysrhythmias) with minimally invasive strategies for specific cardiac diseases.

Aspects of the present disclosure are directed to a hollow tube, which is somewhat flexible and generally of a diameter of 10-20 mm. The hollow tube has a sufficient length so that a distal end of the hollow tube can be placed underneath the human posterior left atrium, with a proximate end of the hollow tube being external to the body and having a general long axis length of 20-30 cm. Within the hollow tube is a secondary, smaller caliber hollow tube that runs along the entire long axis of the hollow tube, and is meant to accommodate a flexible videoscope, such as a bronchoscope. The hollow tube is gently curved in its distal quarter length, so that the hollow tube is generally J-shaped in construction. This curve allows the cannula device to approach and access cardiac structures that would not be accessible with a straight tube, particularly with the limitation of sub-xiphoid access of the heart and pericardium underneath the xiphoid access.

Also contemplated are mechanisms to manipulate the shape of the cannula device, and especially a distal aspect of the cannula device, which may be deflected in a particular direction or whose “J-shape” is manipulated into more aggressive or gentle curves in order to facilitate intra-pericardial procedures. The cannula device is modular such that an obturator fits within the large hollow tube and may have a groove to allow a lock-and-key fit with the secondary inner hollow tube. The purpose of the obturator is to un-flex the gentle J-shaped curve at the distal aspect of the hollow tube, and render the hollow tube into a straight shape. The obturator also has a blunt and smooth distal tip that can protrude through the hollow tube. The smooth blunt tip and straight shape of the assembled obturator and hollow tube allow for safe deep deployment of the assembled cannula device deep into the space between the heart and diaphragm using sub-xiphoid access. If the tube is curved or the open-end of the hollow tube is not protected with a blunt/smooth cover, an inferior portion of the heart can be damaged during placement.

Once the assembled cannula device is placed deep within a heart sac (between the heart and diaphragm), the obturator is removed, and the hollow tube assumes the J-shaped curve and can be safely advance underneath the heart and the left atrium specifically. A videoscope or bronchoscope is placed into the secondary inner hollow tube, which is generally of caliber 4-8 mm. The scope is used to provide video guidance for procedures under the heart such as ablation of the posterior wall of the left atrium. Other ways to use this access cannula device utilize sub-xiphoid access as well, and placement of the cannula device between the anterior surface of the heart and the pericardial (heart) sac. More specifically, aiming the cannula device (hollow tube) towards the left shoulder, and then flipping the J-shaped curve 180 degrees will allow videoscopic access to the left atrial appendage, which can then be ligated (excluded from the circulation) using existing devices for that purpose. Similarly, placement of the hollow tube between the surface of the left ventricle and pericardium (heart sac) will create space to map ventricular rhythm disturbances and allow visualization of ablation using standard tools. While the cannula device described may be a hollow cylinder, other shapes may be advantageous for sub-xiphoid access and for trans-pericardial procedures in general, specifically oval or flatter-shaped hollow tubes that otherwise share similar characteristics as described above.

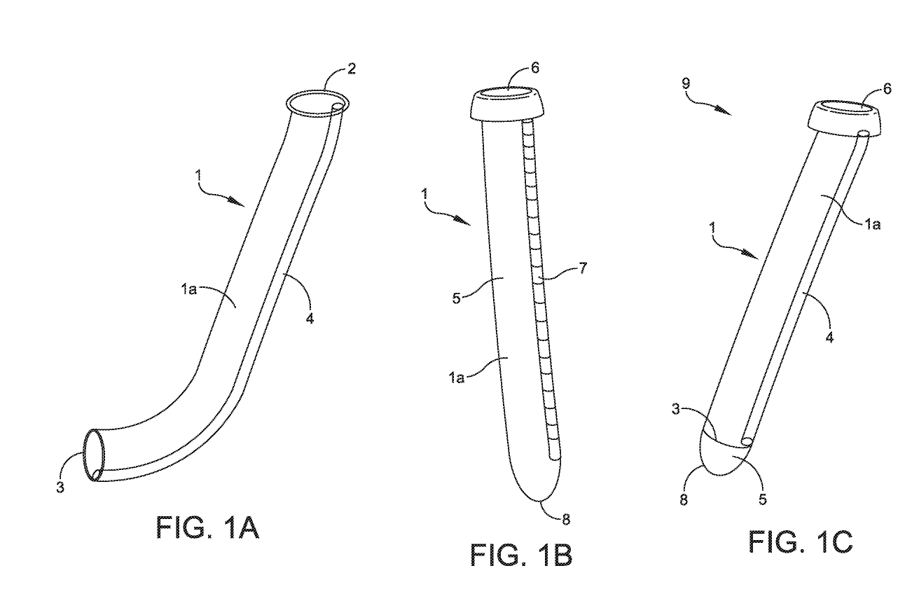

Turning now to the drawings, FIGS. 1A-C are perspective views of a general assembly of a cannula device generally indicated at 1 including a larger hollow tube and a smaller caliber channel or tube within the larger hollow tube, and displaced to one aspect of the larger hollow tube. A distal end of the large hollow tube is gently curved so that it is generally achieves a J-shaped construction. The hollow tube is configured to become straight when an obturator is placed within the distal end of the large hollow tube.

FIG. 1A illustrates the cannula device 1 embodying a hollow tube 1a that is open at a proximate end 2 of the hollow tube and a distal end 3 of the hollow tube. In a certain embodiment, the hollow tube 1a has a cross sectional area between 80 and 300 mm² or a diameter 10-20 mm, and long axis length of 20-30 cm. The hollow tube 1a assumes a generally J-shaped curve towards the distal end 3 of the hollow tube, e.g., the outer quarter length of the distal end of the hollow tube. Within the access cannula device 1 is a secondary hollow tube or channel 4 that extends along a long axis of the hollow tube 1a of the access device 1, and also follows the general J-shaped curve in the distal end 3 of the hollow tube. In other embodiments, the secondary hollow tube or channel 4 may be provided along an outer surface of the hollow tube 1a or within the wall of the hollow tube itself.

FIG. 1B illustrates an obturator 5 that fits over and within the open end 3 of the hollow tube 1a of the access device 1. The obturator 5 has a proximate end cap 6 and a smooth blunt distal end 8. The obturator 5 also may have a groove 7 that is concave, and can accommodate a displacement of secondary hollow channel 4 (see FIG. 1A).

FIG. 1C illustrates an assembled modular device 9 with the obturator 5 placed over the distal end 3 of the access device 1. The end cap 6 stops the obturator 5 in place and distal smooth blunt end 8 protrudes out of distal end of hollow tube 1a. The secondary hollow channel 4 is nestled within obturator groove 7. The obturator 5 has a blunt or rounded distal end and the entire obturator fits within the hollow tube 1a, with the distal blunt end of the obturator protruding through the hollow tube. The end cap 6 cannot pass through the hollow tube 1a because it is larger than the opening of the hollow tube.

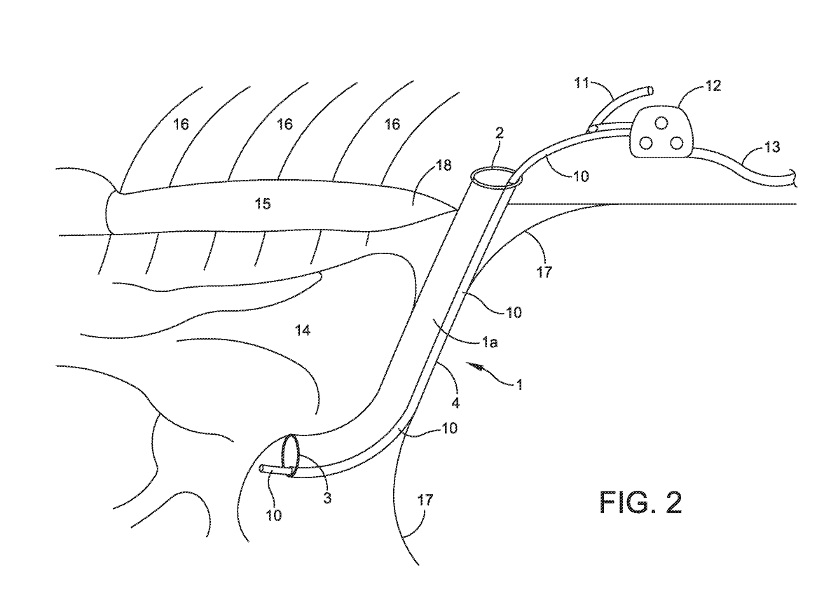

Referring to FIG. 2, the cannula device 1 is used by placement in a sub-xiphoid location between a bottom of a breastbone and abdomen. The gentle J-shaped curve of the large hollow tube 1a of the access device 1 allows the tube to be advanced underneath the heart, or into other remote locations (not shown) that would be facilitated by the J-shaped curve. In this drawing figure, a videoscope or bronchoscope (a “scope”) is placed into the secondary inner hollow tube or channel 4, and the scope is connected to a power and video display to facilitate the procedure and also typically has a channel for infusing or suctioning fluid out of the field.

In one embodiment, the access device 1 is passed below a sternum 15 (and attached ribs 16), and its lowest point, the xiphoid process 18. The access device 1 has been passed through the surface of the pericardium (heart sac, not shown), between a diaphragm 17 and a heart 14. As shown, the obturator (not shown in FIG. 2) has already been removed, and the distal end 3 of the hollow tube 1a of the access device 1 has assumed a J-shaped curve. The distal end 3 of the access device is directed under the left atrium of the heart 14. A flexible videoscope 10 has been passed through the secondary inner hollow channel 4, and protrudes slightly beyond the distal end 3 of the access device 1. The flexible scope 10 is attached to its camera and controller 12, which is further attached to monitors and power sources via cable 13. Integral to the flexible scope 10 is a channel 11 for instilling or suctioning fluid via the flexible scope.

In certain embodiments, the cannula device 1 is modular in construction, such that the obturator 5 fits within the large hollow tube 1a and has a groove to allow a lock-and-key fit with the secondary inner hollow tube 4. The purpose of the obturator 5 is to un-flex the gentle J-shaped curve at the distal end 3 of the hollow tube 1a, and to render the hollow tube into a straight shape. The smooth blunt distal end 8 and the straight shape of the assembled obturator 5 and the hollow tube 1a allow for safe deep deployment of the assembled device 1 deep into the space between the heart and the diaphragm using sub-xiphoid access.

Once the assembled device 1 is placed deep within the heart sac (between the heart and the diaphragm), the obturator 5 is removed, and the hollow tube 1a assumes its J-shaped configuration and can be safely advanced underneath the heart and the left atrium. The videoscope 10 is placed into the secondary inner hollow tube 4, which is generally of caliber 4-8 mm. As mentioned above, the videoscope 10 is used to provide video guidance for procedures under the heart 14, such as ablation of the posterior wall of the left atrium. While the cannula device 1 described may embody the hollow tube 1a, other shapes may be advantageous for sub-xiphoid access and for trans-pericardial procedures in general, specifically oval or flatter-shaped hollow tubes that otherwise share similar characteristics as described above.

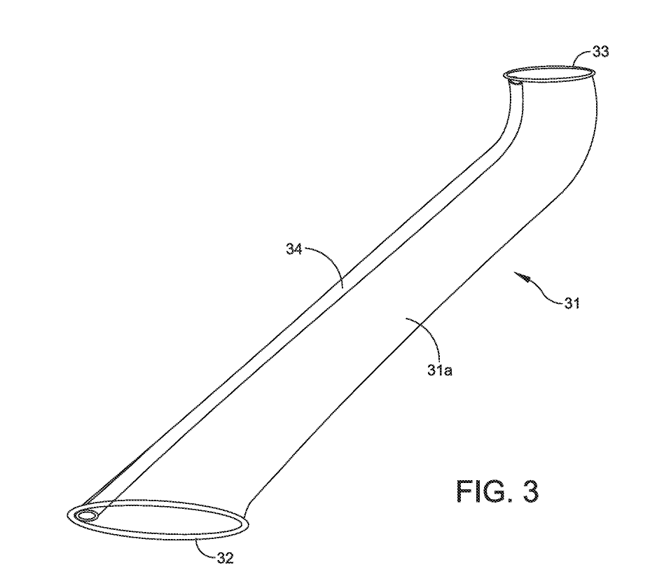

Referring to FIG. 3, a cannula device generally indicated at 31 of another embodiment of the disclosure has a different shape, more specifically a flatter or ovoid shape that may be better for navigating this cannula device into tighter spaces within the pericardium. Specifically, FIG. 3 illustrates a slightly different shape of access device 31, namely where the hollow tube 31a is flatter or more oval shaped, the hollow tube having a proximate end 32 and a distal end 33. The cannula device 31 further includes a secondary hollow inner channel 34, and assumes a generally J-shaped curve towards the distal end 33.

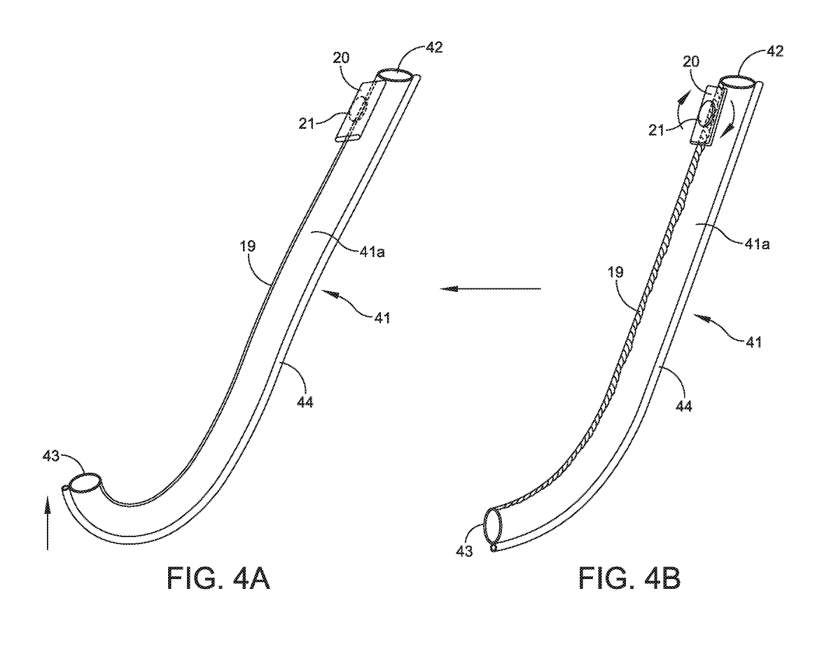

Referring to FIGS. 4A and 4B, a cannula device generally indicated at 41 of another embodiment of the disclosure is deflectable or steerable so that its distal aspect can be actuated to be more or less pronounced and assume a slightly different shape or curve. As shown, the cannula device 41 includes a hollow body 41a having a proximate end 42 and a distal end 43. The cannula device 41 further includes an integrated wire or actuator 19 that runs along the longitudinal aspect of the hollow tube 41a of the cannula device 41. An actuating housing 20 and an actuating knob 21 manipulate and interact with wire or actuator 19 to extend or contract the wire or actuator, and thereby manipulate the shape of the hollow tube 41a of the cannula device 1, which is shown having the distal aspect 3 being deflected upwards to facilitate some aspect of the procedure, i.e., visualization or approach to anatomic structures.

The phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having thus described several aspects of at least one embodiment, it is to be appreciated various alterations, modifications, and improvements will readily occur to those skilled in the art. Such alterations, modifications, and improvements are intended to be part of this disclosure and are intended to be within the scope of the disclosure. Accordingly, the foregoing description and drawings are by way of example only, and the scope of the disclosure should be determined from proper construction of the appended claims, and their equivalents.

What is claimed is:

1. A device for facilitating trans-pericardial or sub-xiphoid cardiac procedures, the device comprising a flexible primary hollow tube with radii of generally 5-15 mm,
wherein the primary hollow tube includes a generally J-shaped curve at a distal end of the primary hollow tube, and
wherein the primary hollow tube is configured to accommodate an obturator that straightens the primary hollow tube and eliminates the J-shaped curve at the distal end.

2. The device of claim 1, wherein the J-shaped curve of the primary hollow tube can be manipulated, exaggerated or lessened by an integral mechanism within a wall of the primary hollow tube.

3. The device of claim 1, wherein the primary hollow tube includes a secondary inner hollow tube having a diameter 4-8 mm, the secondary inner hollow tube extending along a length of the primary hollow tube.

4. The device of claim 3, wherein the secondary inner hollow tube is configured to flex and includes a generally J-shaped curve at a distal end of the secondary inner hollow tube.

5. The device of claim 3, wherein the secondary inner hollow tube is configured to allow passage of a flexible videoscope.

6. The device of claim 5, wherein the flexible videoscope is coupled to a controller.

7. The device of claim 1, wherein the obturator contains a groove to allow the displacement of the secondary inner hollow tube and fits with it in a lock-and-key configuration.

8. The device of claim 1, wherein the primary hollow tube is generally flat or oval-shaped.

9. The device of claim 1, wherein the primary hollow tube has a cross sectional area between 80 and 300 mm2 or a diameter 10-20 mm, and long axis length of 20-30 cm.

10. A method of placing a primary hollow tube with a generally J-shaped curve at a distal end of the primary hollow tube via sub-xiphoid or trans-pericardial access in order to carry out procedures on a surface of the heart.

11. The method of claim 10, further comprising straightening out the primary hollow tube with an obturator to eliminate the J-shaped curve at the distal end.

12. The method of claim 10, further comprising manipulating, exaggerating or lessening the J-shaped curve of the primary hollow tube by an integral mechanism within a wall of the primary hollow tube.

13. The method of claim 10, wherein the primary hollow tube includes a secondary inner hollow tube having a diameter 4-8 mm, the secondary inner hollow tube extending along a length of the primary hollow tube.

14. The method of claim 13, wherein the secondary inner hollow tube is configured to flex and includes a generally J-shaped curve at a distal end of the secondary inner hollow tube.

15. The method of claim 13, wherein the secondary inner hollow tube is configured to allow passage of a flexible videoscope.

16. The method of claim 15, wherein the flexible videoscope is coupled to a controller.

17. The method of claim 10, further comprising securing the obturator in place on the primary hollow tube.

18. The method of claim 17, wherein the obturator contains a groove to allow the displacement of the secondary inner hollow tube and fits with it in a lock-and-key configuration.

19. The method of claim 10, wherein the primary hollow tube is generally flat or oval-shaped.

20. The method of claim 10, wherein the primary hollow tube has a cross sectional area between 80 and 300 mm2 or a diameter 10-20 mm, and long axis length of 20-30 cm.
